(19) United States
(12) Patent Application Publication
(10) Pub. No.: US 2014/0200529 A1
(43) Pub. Date: Jul. 17, 2014

(54) POST-NIPPLE RECONSTRUCTION PROTECTOR

(71) Applicant: Julie Hyde-Edwards, Royal Oak, MI (US)

(72) Inventor: Julie Hyde-Edwards, Royal Oak, MI (US)

(21) Appl. No.: 14/227,863

(22) Filed: Mar. 27, 2014

Related U.S. Application Data
(63) Continuation-in-part of application No. 12/869,809, filed on Aug. 27, 2010, now Pat. No. 8,686,214.
(60) Provisional application No. 61/237,383, filed on Aug. 27, 2009.

(51) Int. Cl. A61M 35/00; A61F 13/14

(52) U.S. Cl. CPC A61M 35/00; A61F 13/14
USPC 604/304; 604/307

(57) ABSTRACT

An improved post-operative dressing for providing both breast and, notably, nipple reconstruction and which in particular establishes custom lateral supporting of the nipple annulus. A body is constructed of a medical grade silicone and exhibits a three dimensional shape adapted for placement over the reconstructed breast and an aperture defined by an inner rim configured within the body is adapted to seat therethrough an associated nipple graft or flap in a laterally supporting and non-pressure applied fashion. The dressing may include a controlled release transdermal delivery system for administering at least one medicinal drug directly to the breast mound and/or nipple.

### CROSS REFERENCE TO RELATED APPLICATIONS

This application claims priority to Provisional Patent Application No. 61/237,383, filed Aug. 27, 2009, and is a Continuation-In-Part of patent application Ser. No. 12/869,809, filed Aug. 27, 2010, now U.S. Pat. No. 8,686,214 issued Apr. 1, 2014, the entire disclosures of which are hereby incorporated by reference and relied upon.

### BACKGROUND OF THE INVENTION

1. Field of the Invention

The present invention discloses a one-piece silicone gel dressing for post-operative use in breast and nipple reconstruction surgeries, and exhibiting a generally arcuate and variable thickness consistency and which is generally shaped to correspond to the contour of the female breast mound. The present invention teaches such a device in which a center cutout and aperture defined portion, in combination with the concave surface of the device, provides a desired minor degree of gentle pressure to the surrounding areola, while concurrently providing lateral support but no direct pressure to the reconstructed nipple and which is permitted to project through the center aperture.

2. Description of Related Art

The prior art is documented with examples of breast reconstruction devices, such as following mastectomy or other breast related surgeries and which are typically geared toward shaping of the breast. A shortcoming of such prior art devices is in the shaping of the reconstructed nipple, with such known applications including clips, collar devices and the like which typically attach directly to the areola, in surrounding fashion to a reconstructed nipple graft or flap. Shortcomings include both discomfort to the wearer, as well as the evident nature of the device when worn under clothing.

An example of one known nipple reconstruction device is set forth in US Patent Application Publication No. 2006/0106329, to Hammer et al., and which including a padded component 12, a core component 14 and an attaching component 16. In relevant part, the core 14 contains a hollow opening 18 for permitting insertion of the reconstructed nipple, however establishes no lateral supporting or other reconstructive aspects not related to the underlying breast mound.

Imonti, U.S. Pat. No. 4,870,977 teaches a surgical protector for raised wounds and which includes an aereola/nipple surface wound protector exhibiting a cone shape. Becker, U.S. Pat. No. 4,790,309 discloses a tissue expander stent for nipple reconstruction and which defines a frusto-conical shaped segment with a short cylindrical tubular element extending outwardly and which encompasses a rigid ring. In application, the conical segment is forced downwardly onto the breast, following which the nipple is sutured to the stent in order that the resiliency of the conical segment maintains the sutures under tension and tends to pull the nipple upwardly during healing.

### BRIEF SUMMARY OF THE INVENTION

The present invention discloses an improved post-operative dressing for providing both breast and, notably, nipple reconstruction which is an improvement over the prior art and which in particular establishes custom lateral supporting of the nipple annulus, combined with a series of secondary advantages including, most notably, invisibility when worn under clothing and, alternatively or accumulatively, each of breathability, suction/adherence, and impregnation or entrainment of any form of anti-biotic (anti-infection) medicine. The body is constructed of a medical grade silicone and exhibits a three dimensional shape adapted for placement over the reconstructed breast and an aperture defined by an inner rim configured within the body is adapted to seat therethrough an associated nipple graft or flap in a laterally supporting and non-pressure applied fashion.

In one non-limiting variant, the outer perimeter of the body is selected from any of including a rounded, oval or ellipsoidal shape and further such that the body exhibits a narrow most and tapered outer profile increasing in dimension to a most thickened interior corresponding to location of the aperture defining inner rim. A tacky adhesive may be applied to an inner concave surface associated with the body to enhance surface gripping onto the surrounding skin. Other features include the adhesive exhibited by a gentle paper tape.

Additional features include a gripping pattern formed into an outer radial edge portion of an inner concave surface of the body and providing additional gripping support against the wearer's skin and about the outer perimeter of the dressing. Pluralities of surface perforations are formed through the body and accessible to a surface in order to improve air permeability. Additionally, the body may include interior molded channels communicating interior located apertures with the wearer's skin, with outer located baffles permitting fresh air to pass through the dressing body to the breast and for generated body warmth to escape through the baffles.

### BRIEF DESCRIPTION OF THE SEVERAL VIEWS OF THE DRAWINGS

Reference will now be made to the attached drawing, when read in combination with the following detailed description, wherein like reference numerals refer to like parts throughout the several views, and in which:

FIG. 1 is a perspective view of the dressing device according to one potential embodiment of the present invention;

FIG. 2 is a top view of the device shown in FIG. 1;

FIG. 3 is an inverted bottom or inside view of the device;

FIGS. 4A-4H are a progression of views detailing the stages of a breast and nipple reconstruction, followed by post-surgical application of the post nipple reconstruction dressing/scar reduction and nipple protector device according to the present invention and further showing the variable thickness of the dressing device including a greatest thickness at a center location corresponding to the reconstructed nipple seating through the center aperture;

FIGS. 5A-5E illustrate a succession of views of the dressing in FIG. 4 and, with particular reference to the side views of FIGS. 5B-5E, the provision of dressings of varying thicknesses and radii;

FIG. 5F is a partial perimeter edge view of a selected dressing and further showing an additional gripping pattern formed into the outer radial edge portion of the inner concave surface;

FIGS. 6A-D illustrate a further variant of reconstruction dressing incorporating a plurality of surface perforations to improve air permeability;

FIGS. 7A-7C illustrate a yet further variant of reconstruction dressing in which interiorly configured aperture patterns are combined with interior defined channels with outer baffles, and in order to facilitate ventilation of the wearer's skin;

FIG. 8 is a use illustration in which the reconstruction dressing is worn invisibly underneath a dress, shirt or blouse;

FIG. 9 a cross-sectional view of a female breast covered by a dressing according to another alternative embodiment of this invention; and

FIG. 10 is an enlarged view of the area circumscribed by 10 in FIG. 9.

### DETAILED DESCRIPTION OF THE INVENTION

With reference to the several illustrations described below, the present invention discloses a one-piece, three dimensional shaped and medical grade silicone gel dressing 10 for post-operative use in breast/nipple reconstruction surgeries. Generally, nipples can be made by grafting skin from another site or using local skin in a flap. As will be described in more detail, the dressing 10 provides a number of improvements not evident in the prior art, including notably provision of a suitable one piece dressing for providing reconstructed breast support in combination with being properly sized for laterally supporting (but not pinching or deforming) the sensitive reconstructed nipple, such dressing 10 further is capable of being worn in a virtually invisible nature underneath any normal feminine garment.

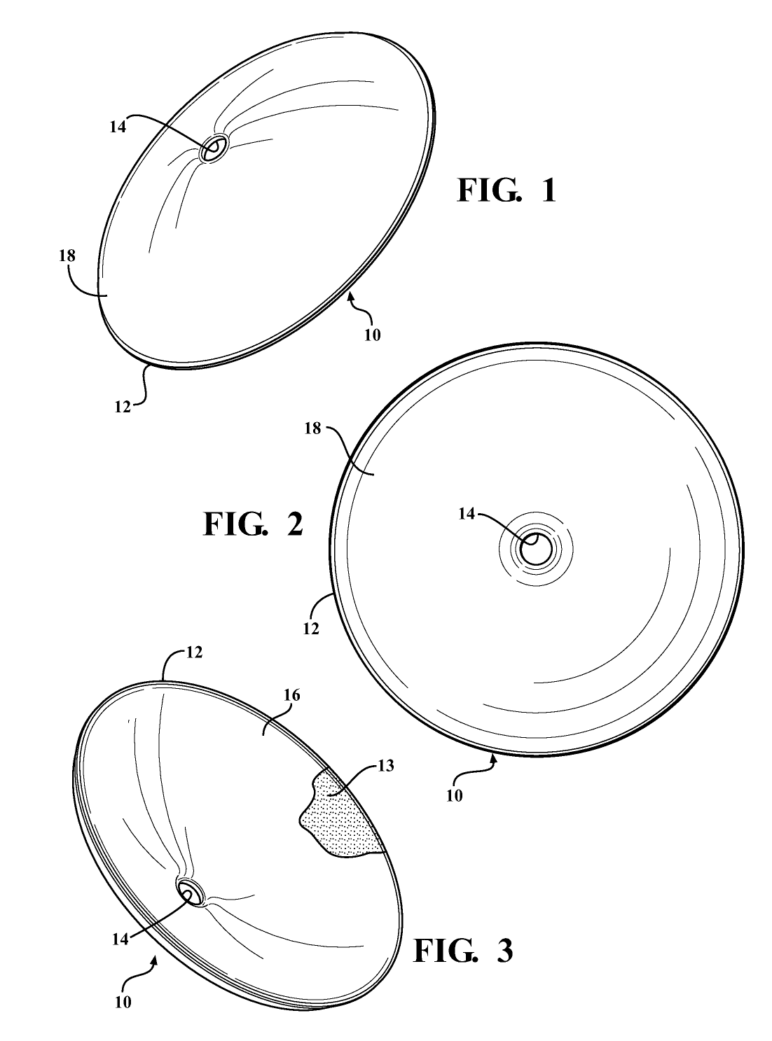

With reference initially to FIG. 1, the dressing 10 is provided as a body, again typically constructed of a medical grade silicone but also contemplating the inclusion of other suitable materials. The body of the dressing 10 exhibits a generally arcuate and variable thickness consistency and which is generally shaped to correspond to the contour of the female breast mound. As shown, the dressing 10 exhibits a generally tapered and narrowest thickness associated with its outermost and perimeter defined edge, at 12, and which can adopt any of a generally round, oval or ellipsoidal shape depending upon the overall contour of the reconstructed breast to which it is applied.

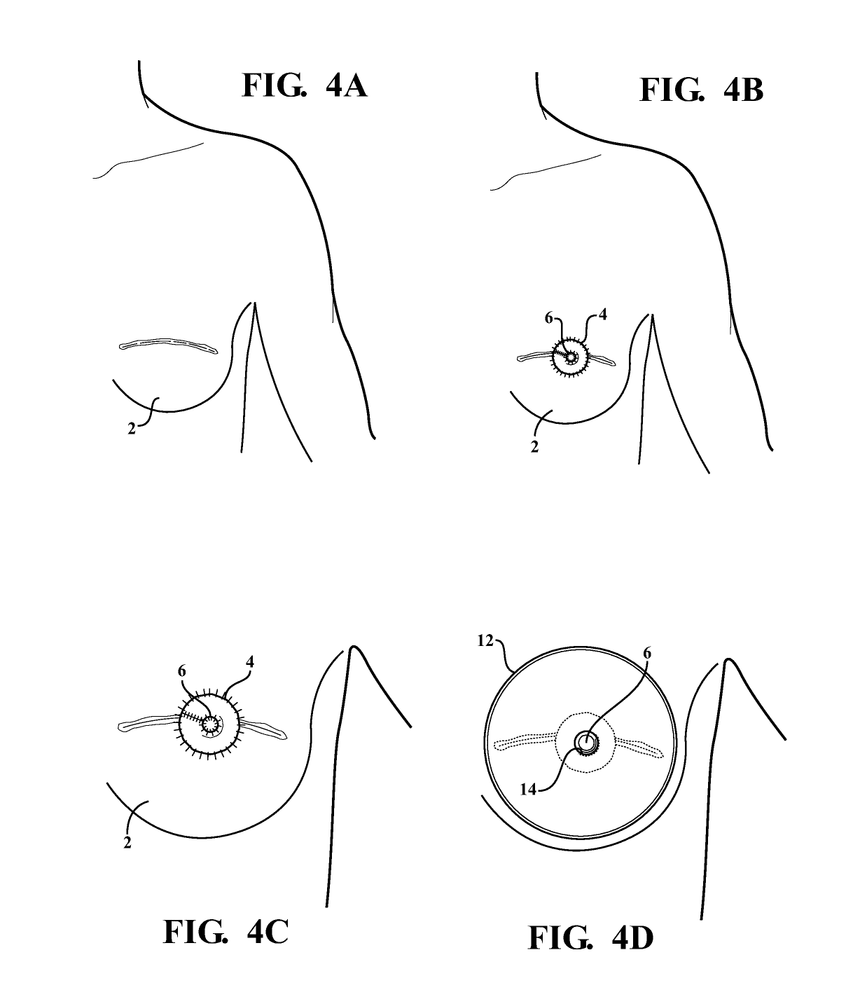

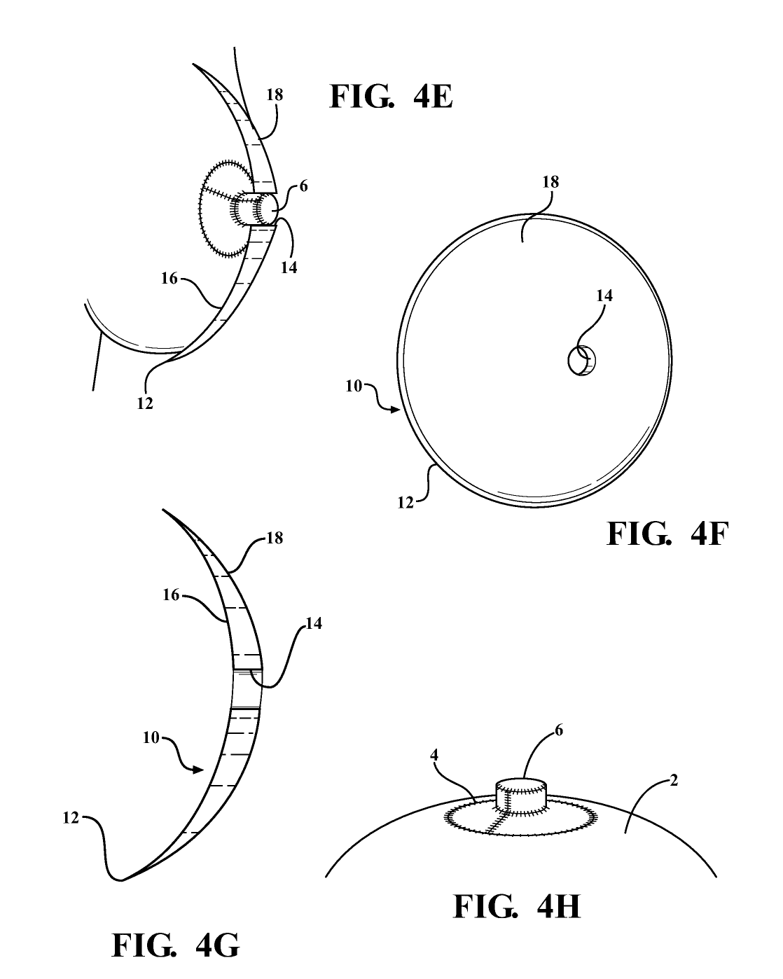

FIGS. 4A-4C and 4H show a progression of views detailing the stages of a breast and nipple reconstruction. FIGS. 4D and 4E depict a post-surgical application of the post nipple reconstruction dressing/scar reduction and nipple protector device 10 according to the present invention. These figures, in combination also with FIGS. 4F and 4G, further show the variable thickness of the dressing device 10 including a greatest thickness at a center location corresponding to the reconstructed nipple seating through the center aperture. The thickness of the device 10 increases to a maximum dimension (such as ½″ in one desired variant) at a generally central location corresponding further to an aperture (see inner annular rim 14 in each of FIGS. 4D-4F).

As is known, and following a mastectomy procedure (see again FIGS. 4A-4C and 4H), a complete breast reconstruction includes recreating the breast mound 2, the nipple 6 and areola complex (NAC) 4, such that the breasts are symmetric with regard to pigmentation, shape, size, projection and position. It should be understood that the areola complex (NAC) 4 and nipple 6 are considered part of the breast mound 2 for purposes of this patent. This is a multi-stage process, with the NAC reconstruction often considered to be the final stage.

The center cutout and aperture defined rim 14, in combination with the overall (inner facing) concave surface of the device (see further at 16 in FIG. 4E), is custom designed in order to provide adequate lateral supporting aspect around the entire periphery of the reconstructed nipple, this further defined as a desired minor degree of gentle pressure when applied to a breast 2, specifically to a surrounding areola 4 associated with a central projecting reconstructed nipple 6 which comfortably seats through the central aperture and is gently and laterally supported by its rim 14).

In this fashion, the dressing 10 provides a desired degree of lateral support to the outer edges of the nipple 6, such as occurring without application of any direct pressure to the reconstructed nipple, which is permitted to project through the rim 14 defined center aperture. Adherence of the dressing 10 to the breast is further facilitated by the use of any tacky surfacing material (such as a gentle paper tape a portion of which is representatively designated at 13 in FIG. 3), as well as any compound or ingredient applied to a corresponding inner concave surface 16 of the body, this including a non-residue application of a material upon the inner surface 16 which provides a requisite degree of continual adhering support to the breast mound 2 and areola 4. It is further envisioned that the molecular composition of the silicone material, combined with a desired texturing (or roughening) of the inner concave surface 16 and natural contact forces of adhesion generated by the body heat of the wearer, can to a large degree establish the requisite degree of holding forces between the dressing 10 and the reconstructed breast mound 2, and without the need for separate adhesive materials.

The dressing 10 is constructed, as previously described in one preferred embodiment, of a medical grade silicone given its durable, cushioning and flexible nature, with its outer perimeter, dimensions, and variable thicknesses being largely dictated by the dimensions and contours of the reconstructed breast 2. As is further known, medical grade silicone is a material which is clinically proven to reduce scarring of tissue and is known to be sold in sheet form for such a purpose. This is particular value in the present application, given the existence of an incision scar associated with the breast reconstruction, the appearance of which is progressively minimized at a faster rate as a result of the application of the post-surgical and reconstructive silicone dressing device.

Given this, it is desired that the silicone cushioned device can be customized (or produced in a wide range of varying shapes, sizes and configurations, to comfortably seat the reconstructed breast and nipple graft or flap of the wearer. It is further envisioned and understood that other suitable materials, such as drawn from other types of polymeric and/or rubberized based materials can be substituted which provide similar cushioning to the breast and areola, with concurrent lateral and non-pressurized support to the wearer's reconstructed nipple.

In use, the overall configuration of the dressing 10, combined with its generally gentle sloping convex outer surface 18 (opposite the inner concave surface 16 as shown in FIGS. 4E and 4F) provides a device which can be easily and invisibly concealed underneath a surgical bra or other garment and held in place by any suitable means including, but not limited to: adhesive (such as a gentle paper tape), a non-residue and tacky application to the inner concave surface 16, natural surface tension and/or tackiness (inherent in many silicone-based materials), as well as also possibly a vacuum sealing aspect as will be subsequently described. Additional advantages of the dressing 10 include its wash-ability, as well of the central and aperture defined rim 14 facilitating evacuation of moisture or perspiration building up along the inside contact surface between the breast mound and areola and the inner concave surface 16 of the device.

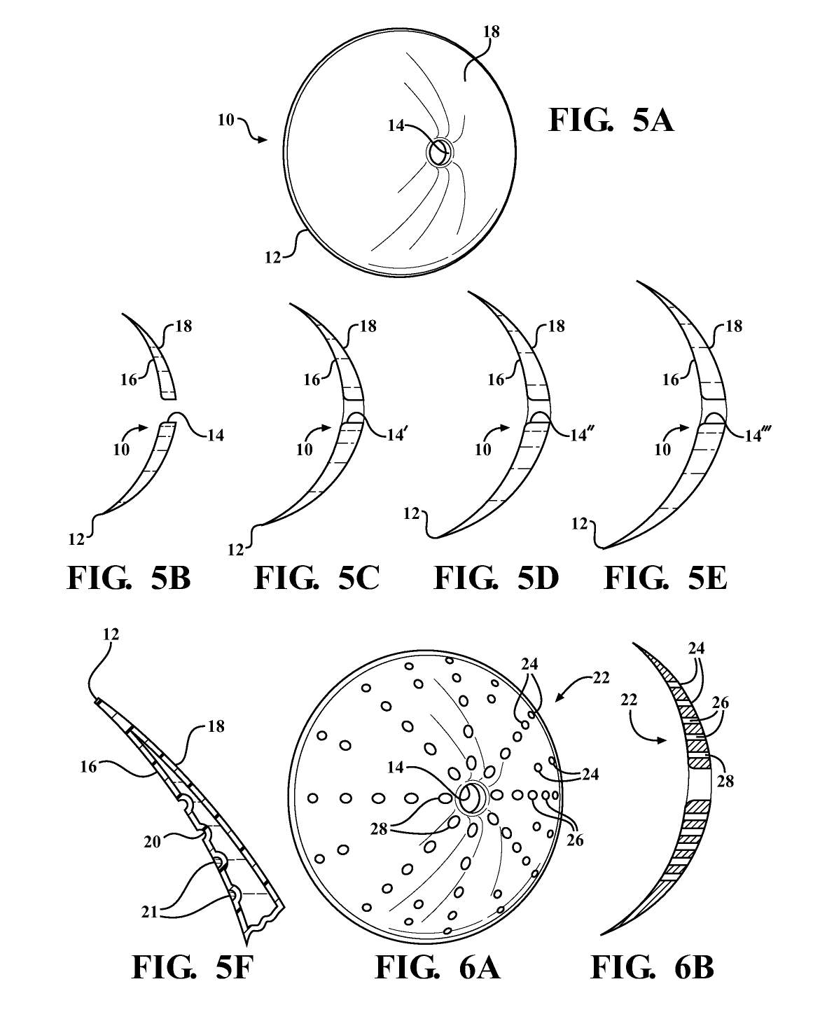

Referring now to FIGS. 5A-5E, illustrated are a succession of views of the dressing 10 in FIG. 4 and, with particular reference to the side views of FIGS. 5B-5E, better illustrates the range and variety of dressings which can be provided according to varying thicknesses and radii. In each instance, the sizing or scalability of the dressing body 10 increases progressively, with the inner nipple seating aperture concurrently incrementing from an original diameter 14 in FIG. 5B, to 14′ in FIG. 5C, 14″ in FIGS. 5D and 14″ in FIG. 5E. One variant of the invention further contemplates providing a plurality of dressings 10 of varying size and nipple aperture (including smaller sized bodies with larger aperture nipple seating locations and vice versa).

Proceeding to FIG. 5F, a partial perimeter edge view is shown of an inner perimeter surface extending in non-linear (or undulating) pattern, see concave depictions 20, of a selected dressing 10 and which further illustrates an additional gripping pattern formed into the outer radial edge portion of the inner concave surface 16, this providing additional gripping (such as warmth induced gripping or other vacuum retaining) support against the wearer's skin and about the outer perimeter of the dressing 10 away from the central and nipple sensitive location.

The concave depictions 20, as well as any location along the smooth inner adhering surface 16 of the dressing, may also include any type of anti-infection or anti-microbial application, this including any form of chemical impregnation either formed into the body of the dressing or (as representatively depicted at 21) inlaid within the concave depressions 20 in order to deliver anti-inflammatory or anti-infection compounds at the particular areas desired. It is also envisioned that the concave depictions 20 can be substituted by any other pattern, scheme or depiction calculated to enhance gripping aspects with or without the inclusion of other adhesive retention support.

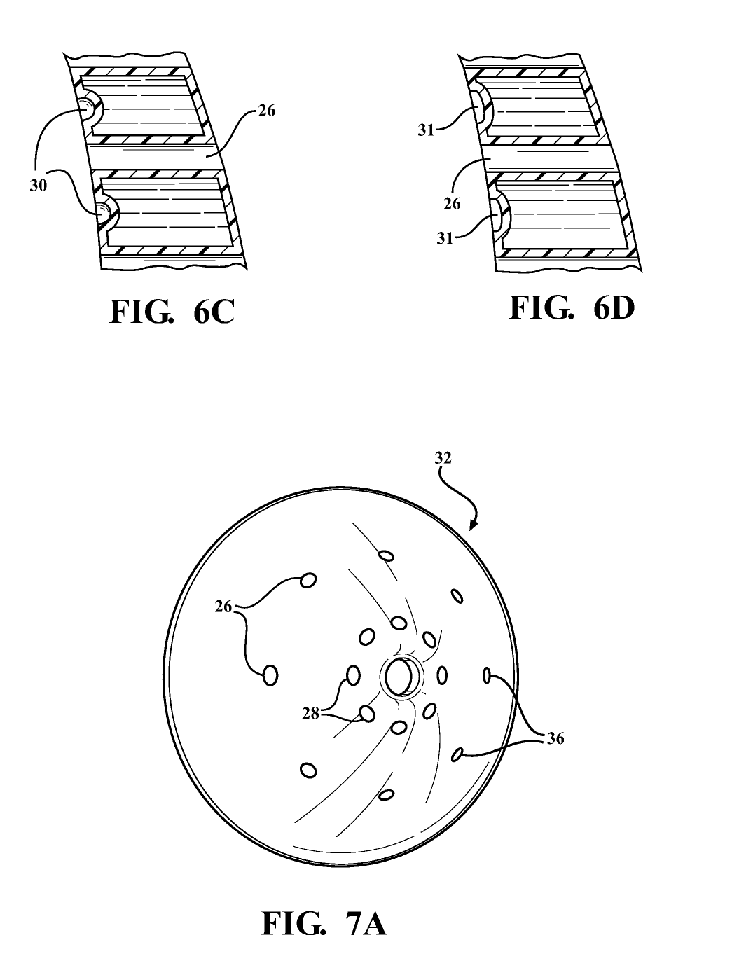

Referring now to FIGS. 6A-D, illustrated are further variants of a reconstruction dressing 22 in each of the front and side cutaway views of FIGS. 6A and 6B, respectively. The dressing body 22 in these embodiments incorporates pluralities of surface perforations or apertures 24, 26 and 28 in a generally outermost to inner extending pattern formed through the surface of the dressing and in order to improve air permeability.

As further best shown in the enlarged partial cutaway views of FIGS. 6C and 6D (taken from FIG. 6B), illustrated are enlarged partial views of the pattern of the apertures 26 is again shown, combined with the formation of additional mini-concave pockets, see as depicted at 30 in a first pattern in FIG. 6C and which is similar to that depicted at 20 in FIG. 5F, and further at 31 in a second and non-limiting alternate pattern in FIG. 6D which is again defined along the interior concave surface of the dressing.

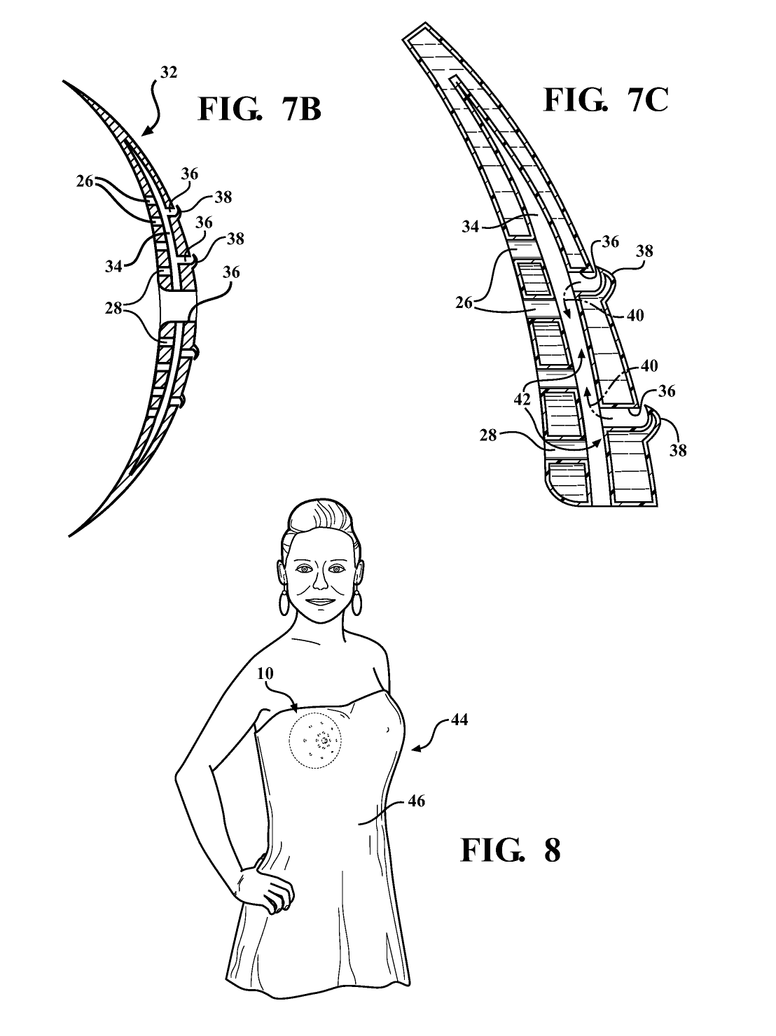

With reference to FIGS. 7A-7C, a series of perspective, side cutaway and enlarged partial cutaway illustrations are progressively shown of a yet further variant 32 of reconstruction dressing, and in which any desired pattern or arrangement of interiorly communicating apertures, such as selected from any combination of apertures and including those in positions as also depicted at 26 and 28 in FIG. 6A. As further shown in FIGS. 7B and 7C, the apertures 26 and 28 are internally communicated by channels 34, and which are in turn also communicated with outer baffles (including apertures 36 over which are positioned partially obstructing and projecting covering elements 38), this in order to facilitate a degree of controlled ventilation of the wearer's skin. In operation, the detail shown of the channel (FIG. 7C) allows fresh air to pass through the body (see directional arrows 40), while allowing the warmth of the body to escape (additional directional arrows 42). In this manner, it is envisioned that the skin surface of the wearer can be ventilated with a dressing 32 incorporating the interior configured (e.g. molded) channels 34 and outer communicating baffle channels 36.

Finally, FIG. 8 is a use illustration, generally at 44, in which the reconstruction dressing, such as previously shown at 10, is illustrated being worn invisibly underneath a dress 46, and which can be equally applicable to being worn underneath a shirt, blouse or any other female upper body garment. The ability to provide a contour establishing and invisible post-operative dressing 10 (or 22 or 32), suitably worn with existing clothing 46, has been found to be a desirable improvement over cumbersome prior art dressing solutions.

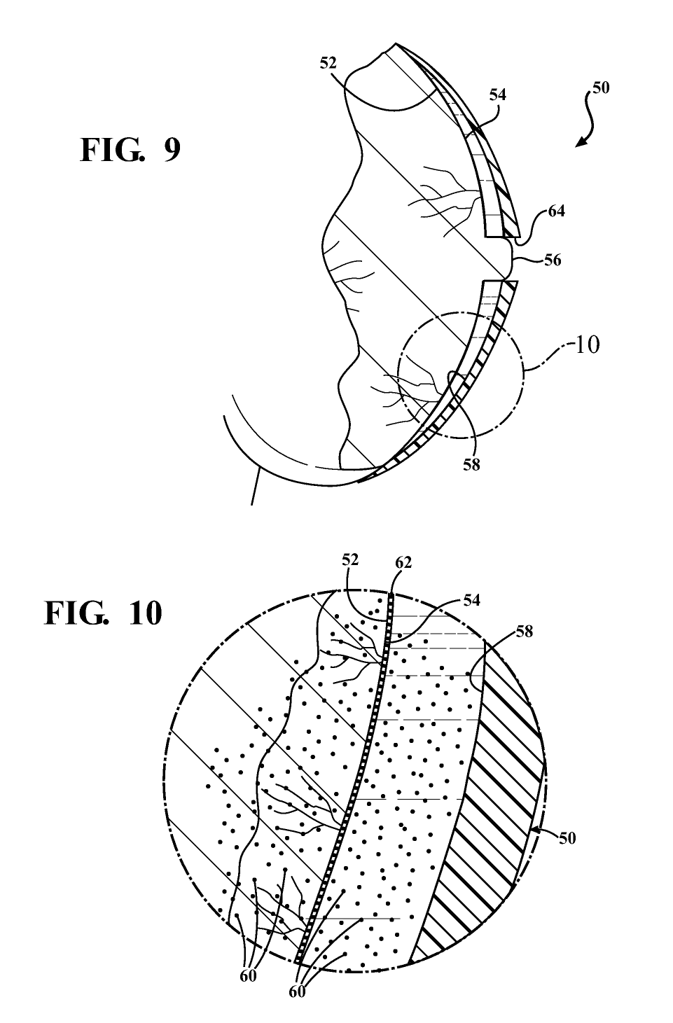

As stated above, the dressing 10 (or 22 or 32) may be constructed of a medical grade silicone to take advantage of that material's known scar-reduction properties, and further still may be chemically impregnated in order to deliver anti-inflammatory or anti-infection compounds to needed areas. FIGS. 9 and 10 further define and illustrate this alternative embodiment of the present invention with a dressing, generally shown at 50, that is configured to provide an efficient vehicle for drug delivery in topical formulations concurrently with the above-mentioned nipple reconstruction benefits. Silicone materials are known for use in pharmaceutical topical formulations. Some common silicone types suitable for pharmaceutical applications include cross-linked silicone gels, liquid silicone rubber elastomers (LSRs), and high-consistency rubber elastomers (HCRs) to name but a few. In all cases, the large atomic volume of the silicon atom and the size and position of the applicable pendant groups result in bond angles that yield a high degree of free volume. This free volume then provides what may be considered “microporous pathways” for liquids and gasses (including water vapor) to migrate through a cured silicone medium so that drug agents can move through silicone. As a direct result, silicones are well suited platforms for drug delivery.

The prior art has taught both tin catalyzed and platinum catalyzed silicones for use in drug delivery applications. In some cases, the silicone material is combined with various pharmaceutical agents as useful antibiotics, antidepressants, anxiolitics, antifungals, antivirals, analgesics, and permeable to selected vitamins. Many pharmaceutical infused silicone materials can release 10 to 500 micrograms of agent per day, and are well-suited to the delivery of anti-inflammatory, anti-infection and other desirable compounds to areas of a human breast 2 in post-operative care.

Any suitable drug may be infused in the body of the material in the form of a homogenous matrix or be stored in a reservoir separated from the patient's skin by a drug release membrane. In a matrix design, the active drug agents are mixed homogenously into the silicone and then molded, extruded, etc. into the desired geometry. One example of a matrix design may be seen in a central venous catheter impregnated with drug agents intended to combat infection. Reservoir configurations, by contrast, concentrate the active drug in a void formed within a molded silicone part. The illustrations of FIGS. 9 and 10 depict the present invention in the context of a reservoir configuration. Both matrix design and reservoir type configurations possess inherent advantages and shortcoming, such that one may be preferred over the other depending on design considerations, the particular chemical characteristics of the drug agent, manufacturing concerns, treatment objectives, and the like. The specific silicone material composition can also be tailored to some extent to achieve the desired results.

The applications are many for the present dressing 50 configured for concurrent drug delivery. It is well known that immediately post-surgery, a patient's risk of infection is generally greatest. By using this dressing 50 infused with an anti-biotic drug, the shaped inner concave silicone surface 52 (and/or the inner rim of the nipple aperture 64) will come in direct contact with the new incisions on the breast 54 and help prevent infection while concurrently protecting the reconstructed nipple 56. Scarring is another common issue after breast reconstruction and nipple reconstruction. The dressing 50 can be infused with wound healing drugs to deliver the drugs directly to the surface of the scar as it heals. In cases where a patient's skin does not heal well, the dressing 50 can be infused with growth factor drugs to deliver the drug directly to the wound and aid in healing. Some patients are prone to variations in skin pigmentation at the site of incisions. In these cases, the dressing 50 may be infused with anti-pigmentation drugs delivered directly to the incision while protecting a reconstructed nipple. In the case of inflammatory breast cancers, patients often have ulcers or metastases on the breast surface. By infusing the dressing 50 with an anti-neoplastic drug, the drugs are delivered directly to the surface of the breast 54 where needed without interfering with the nipple reconstruction 56.

In FIGS. 9 and 10, the dressing 50 is shown constructed in the one exemplary mode of a reservoir type configuration. In the illustrated case, an internal reservoir 58 contains a quantity of drug agent 60 suspended in a suspension medium or otherwise presented for use. The reservoir 58 is located between the convex outer surface and the concave inner surface 52 of the dressing 50. If the suspension medium is sufficiently stiff, it may be in open and direct contact with the patient's skin. Otherwise, if the suspension medium is viscous or liquidy, the reservoir 58 may be covered with a drug release membrane 62 that rests in direct contact with the patient's breast 54 (including the nipple 56). According to well-known processes, the drug agents 60 migrate at a design release rate into the patient's skin tissue, either directly from the suspension medium or through the membrane 62. This is shown in the enlarged cross-sectional view of FIG. 10, in which a drug release membrane 62 covers the reservoir 58 and forms, at least partially, the inner concave surface 52 and/or the interior surface of the inner rim 64. Although the treatment area is shown in FIG. 9 to encompass substantially the entire inner concave surface 52 and of the inner rim 64, the reservoir 58 could be strategically sized/located to deliver drug agents 60 to selected areas of the patient's breast 54 and/or nipple 56. Alternatively still, the dressing 50 could be configured with multiple discrete reservoirs containing different drug agents to accomplish a more complex treatment protocol. That is, different drug agents can be delivered trans-dermally to different areas of the breast 54 and nipple 56. It will be understood that a matrix configuration is equally viable to bring about all of these scenarios.

Those of skill in this art will appreciate that the need to apply topical medicines are often at odds with the need to protect a reconstructed nipple 56 while it heals. That is, the act of gaining access to treatment sites on the breast 54 and applying needed medicine—perhaps several times a day—can disturb the nipple 56 and adversely affect the ultimate quality of the surgical reconstruction procedure. The disturbance factor can be compounded in situations when caregiver assistance is required to apply the medicine and/or re-dress the wounds. The present invention, made from a suitable drug-infused silicone material, effectively self-administers the needed drugs to the underlying skin without removing or even disturbing the dressing position so that the nipple 56 can heal substantially undisturbed.

In the case of breast reconstructions, the dressing 50 fits comfortably over the breast 54 while supporting the reconstructed nipple 56 in the full-surrounding manner what may even include the trans-dermal delivery of drug agents radially inwardly about the circumference of a nipple 56. As shown, the dressing 50 can be configured to deliver certain classifications of drugs trans-dermally that may aid in the treatment of scars, uneven pigmentation, prevention of infection, etc. The shape of the dressing 50 is thus unique and specific to the female breast 54 and therefore an ideal device to be infused with treatments and therapeutic drugs for these contemplated applications.

The dressing 50 may be utilized to carry out a controlled release transdermal delivery system for administration of various drugs to the human breast 54 even in situations were the nipple 56 has not undergone reconstructive surgery. In these instances, the silicone form dressing 50 is worn over the breast mound 54, and may be configured with or without an inner rim 64 to allow a natural nipple 56 to pass through. The inner concave surface 52 can be provided with an adhesive skin-contacting layer or tacky drug release membrane 62, or rely on the natural tackiness of the silicone material, to secure the dressing 50 in position on the breast mound 54.

The dressing system of this invention is thus designed to administer various drugs to the breast mound 54 (including the NAC and nipple regions) via the concave inner surface 52. That is to say, in both breast/nipple reconstruction and non-reconstructive surgery applications, a controlled release transdermal delivery system is operative through the concave inner surface 52 and/or the interior surface of the inner rim 64 for administering at least one medicinal drug directly to the breast mound 54 without removing the dressing 50 from the breast 54. The controlled release transdermal delivery system may be any suitable type, including matrix and reservoir types, for administering medicinal drugs. Examples of drugs to deliver transdermal via the dressing 50 may include: hydrocortisone for scar modification; growth factors for wound healing; antibiotics for infection prevention; hydroquinone for anti-pigment; and cur-cumin for anti-neoplastic to name but a few.

Having described my invention, other and additional preferred embodiments will become apparent to those skilled in the art to which it pertains, and without deviating from the scope of the appended claims. This can include the use of any material composition within the dressing body, including again but not limited to medical grade silicone, as well as the construction or addition of any adhering, ventilating, medicating or supporting features to correspond and support the provision of the desirably dimensioned and laterally supporting central aperture for seating the reconstructed nipple therethrough.

What is claimed is:

1. A dressing for post-operative use with a reconstructed nipple, the dressing comprising a flexible silicone body of three-dimensional shape including a convex outer surface and a concave inner surface, the concave inner surface is adapted for placement over and in adherence to a breast mound associated with the reconstructed nipple, wherein the flexible silicone body has a narrow-most tapered outer profile increasing in dimension to a most thickened interior so that the body is shaped to correspond to the contour of the breast mound so as to be rendered generally imperceptible when worn under an upper body garment, and including a controlled release transdermal delivery system operative through the concave inner surface for administering at least one medicinal drug directly to the breast mound concurrently with the flexible silicone body placed thereon and adhered thereto.

2. The dressing of claim 1 wherein the controlled release transdermal delivery system includes at least one drug agent stored in a reservoir between the convex outer surface and the concave inner surface.

3. The dressing of claim 2 further including a drug release membrane covering the reservoir.

4. The dressing of claim 2 wherein the reservoir comprises a first reservoir, and further including a second reservoir containing a different drug agent.

5. The dressing of claim 1 wherein the controlled release transdermal delivery system comprises at least one drug agent infused in the flexible silicone body as a homogenous matrix.

6. The dressing of claim 1 further including an aperture defined by an inner rim configured within the most thickened interior of the body and adapted to seat therethrough the reconstructed nipple in a laterally supporting and non-pressure applied fashion, said controlled release transdermal delivery system operative through said inner rim for administering at least one medicinal drug directly to the nipple.

7. The dressing of claim 1 further comprising a tacky adhesive applied to the inner concave surface.

8. The dressing of claim 7, wherein said adhesive includes a paper tape.

9. A dressing for human breast having a nipple, the dressing comprising a flexible silicone body of three-dimensional shape including a convex outer surface and a concave inner surface, the concave inner surface is adapted for placement over and in adherence to a breast mound of the female breast, wherein the flexible silicone body has a narrow-most tapered outer profile increasing in dimension to a most thickened interior so that the body is shaped to correspond to the contour of the breast mound so as to be rendered generally imperceptible when worn under an upper body garment, an aperture defined by an inner rim configured within the interior of the body and adapted to seat therethrough a nipple of the breast, and including a controlled release transdermal delivery system operative through the inner rim of the aperture for administering at least one medicinal drug directly to the nipple.

10. The dressing of claim 9 wherein the controlled release transdermal delivery system includes at least one drug agent stored in a reservoir between the convex outer surface and the concave inner surface.

11. The dressing of claim 10 further including a drug release membrane covering the reservoir.

12. The dressing of claim 10 wherein the reservoir comprises a first reservoir, and further including a second reservoir containing a different drug agent.

13. The dressing of claim 9 wherein the controlled release transdermal delivery system comprises at least one drug agent infused in the flexible silicone body as a homogenous matrix.

14. The dressing of claim 9 wherein the inner rim has a generally cylindrical shape so that the controlled release transdermal delivery system administers the drug agent radially inwardly about the circumference of a nipple supported therein.

15. The dressing of claim 9 further comprising a tacky adhesive applied to the inner concave surface.

16. The dressing of claim 15, wherein said adhesive includes a paper tape.

17. A method for administering at least one medicinal drug to a human female breast, the method comprising the steps of:
covering a breast mound of the female breast with a flexible silicone body of three-dimensional shape, the flexible silicone body having a convex outer surface and a concave inner surface, the concave inner surface adapted for placement over and in adherence to the breast mound, and
releasing at least one drug agent transdermally into the breast mound through the concave inner surface, wherein said releasing step is concurrent with said covering step.

18. The method of claim 17, wherein said covering step includes laterally supporting a nipple of the female breast in an aperture.

19. The method of claim 17 wherein said releasing step includes passing the at least one drug agent through a drug release membrane disposed in direct contact with the breast mount.

20. The method of claim 17, wherein said releasing step includes transdermally transferring a plurality of different drug agents, each drug agent directed to a different region of the breast mound.
